SEQUENCE LISTING (24 sequences)

SEQ ID NO: 1 - DNA, 5216 bp, unidentified
  source 1..5216: mol_type = genomic DNA
agcgtcgtcgaggtctggaagtgtctgacaagtgccctctctgtgctttggagccggaggacacgttccatgcttttttgcaggtgcccgctggcggtggcgctctggcaaaccatggcggaccaatggcgaatcccggatgtcgcatcattccgccggacgggatcggagtggcttgcccaagccctttgcgatctgccggatatggagaggatggagctcatgatgactttgtggcgatgttggttcgttcgcaacgagctggtgcaccacaagaagccgccgccgattgaagtgtccaaacgcttcctcaccagctacgtcgactctttggtggggattcagaacaatccagaggcgagtctcaccaaggggaagcatgtggttgatacgattgtccccaaacggatcgtgcaacgggaagtccaagcgccgccagtgccgctgcactggtccagaccagccacggggtggacaaagctcaacattgatggatcctttagtgccacgggtggagaggccggagcaggcatgatcgttcacagcgaaaccggtgatatcatcttctcctcctgcagggaactacggatgtgctctgagccccttgaggccgagctacatgcatgcatggagggctcaatctagcactgtagtggactctgttgcctattgtgatggaaacggactgttcggtggcactacatgcgatattagctccagtgccggacagatctcgttttgctatgcttgtcgaccaggttagacgcttgatgccaggaggtagagaaataaagatggttcatgtatgtagagaggagaatagggtaagccactatcttgcgaactacggtagagttcataaacgcatgattgtgtggctgggatcgggaccgaagaaggtcccagatttgtgtaaggccgaggccctttatgtgtgagaaatgaaatttctttcacccgcaaaaaaaagagatcaaatacatgaagataggcataggcatgtggactttacttacaacattatagttgcacttaggatcttctcttaatttccaatatggtgatgtggtctcaacaattagatatggggaatgcgtccaaataatatcacaaccaatcaacaacctacatcgtgagtctattgtccaaacaaggcccttaccgtcacatggggttacgtcgattattaaggatttctccaagccaacacaattggcgttcagtgactacacctcatgattccttgttaggtcttttcggctaccgaaccaatccttactttcataggcgttggtgtaaccttctacgtgtccctgcactttatttcctcctacgttttttggatgcaatggaaaaaagtgctccctgtggttttttttgggtggatcgggcatgacgaaggtccggatacccgtaaacctctcctacatttggttttggtttgtgtgattttagactccagtatggtttggacaaatttaatgacccacgttgacaaaatatttggaccattcggtctacacatttaaaggcgatttggtgaaccacattgaagttgccgttacaagggtgaatcgttttgttggatgcgttataccgttcgccagctagggttgacatttgtgaaccgcctagattgcagtttaagtgtgcttggggacggcagttttacaaaaggcatggcattttatttattttagtgcattttcatctaaaaactgccaccatcaaatcttgatcgtgtggcgtgtggaggggttgctggagggatgttcctggcgactgttcgccaggtaataacattgccaaaaagaaaaaaaaaagcagtagagcgtgattttgagccgcggtacaaaggtacccgtagcaggttgcccctatccgtatccggtcgcctaaatattttcctccctgtcgccctccctccttcctttcctccactcgcgcttcgctctcagatcccttctcctccgtctcgcccgcgtgttggggtaggagccatggcggaccagctcaccgacgaccagatcgccgagttcaaggaggccttcagcctcttcgacaaggacggagacggttcgatcccttctctgttctctttcaacatgtactactatatgatttgcacgactccattcgatgtgcgcgcactcgctgtggttgatgcggcgccttgcactggcgcgtggatgtgttgggtgggagatctgatcgacgtagcgtatatgcgtttgctggtccgagtggatcgcggcgggattggtttgcggggtgctcaagatgcagtagatcttgcattccttttctccactccttgtggattctggttccatacattcttgtcttgccagcgctatcaacccgtcaccgtccagtgttctggtattgctgcgccttatgctcgtccgttccggctgatcgctgagctgtctgtcatagcgaaggcttcagccggctgtttggtcgttccttcagatctctttcagtggagcaactgatgaccttgcaaagttccaaaaaaaaaaggttctactaggaagagattgaattatgagagaaggtggcatcatggggttgctgcggttagcatgggtctaccgcccacacatctagtggtgtctgcagcttaagaaaaccgagggtgcagcatgggtgatcacacagcatggccggtggcactgttgctgctgagtaggttgctagagcatggtgggtcactagtcatcagtctctactctagatttagctggaacagcagtgacgaggtgtgctgcgaagtggtgttaccgtgttagggttataggatcatgtaatacccataattctgtacttgtatgttagtaatggggaactactactgacttggaatttatctaggcttttgggtctttttctatggatgacatgggcatagaagtgggatagcatgattaaattttgcgttgctcaaactgcacattttcttctcagtgaatgcaacatcggagtttctctggttttgcctatcatgctatggtggtgctagtgtgagactgataacatggagaatgcatgtgatggcttgaaaagttattggtattgatgtttactgattgtatgaaacattagttgaagtaatttacatgttaccagcttactgattgacagaaaatatatctgtaagttgcttactgattgacatgcaacctattacagccaagcaaatctttgctataataggttgcatgtcttactcttctatttatttgggctttggtactgatcaagtcgatggaatgtttacccaaatacttttctggatagttgtgttccagatagtcaagccttgaatgttcttatgaaatttcatcattgtccacttaaactgtatttgtatttatttgctacccttgaatgatttcatgcacccaaaattccagatgaagaaagggactgtaagatttgttctgatggacccaatcattggtccatattgtcttttgcattagcatagctgaaatgatctctaatggttaaataaatgtaaataatatttgagtccaactgtccattgaaaacccccaagttcgttatgttcttgtactgcacatccatgtgcaaatgtggcttataaatttctataagatcattttgaagattcgcccattactggcacatttcatatagaatacgtgcagcattttacatatgatatgatgattttattgcacaattacttgtgtttttctgcatacttgtgggtttctgtattcccatctaatagtcagtgtttcaagtgggcaagtcgaatcaagttgcacctgactggccaggtgactagtctgactatagatgctgactagtcagtgactatttgacaattagctaggcttgcatagtgaggccatttagacagctggcaataggttttcaagtcatacttggtgtgtctggtttgagcccaagccaaaccccaccaaaaatttggcttaggttttgtctgcccatgttttggccactgctggcaagaaaattgtaagtgggctaaggggcatggacgcatggtcatgccaaatttttgtcagccatccaagcaagaaccaaaattcagtcataaccaaaacaatcattaggtttgatttggttgcaaacaaaccattcccatcttccatgttaagacctcttttattgtgcacagaattgaattgctgacataaatctccggggcatttcattctagcacaatattaccattgtatatatatgagcaatgctacacctacgtaaagctgccatacgtgattaacgtaacagcctaggtggcatatctttgttggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtttacgtaggtttagcattttcgtgtatatatttgtagcgtttttgtatatatatttgtagaagaagtttaaattgttagactaaaaaactgtctgcctcctgaacaggctgcatcaccaccaaggagcttggaactgtgatgcgctcgctggggcagaaccccactgaggcagagcttcaggatatgatcaatgaagtggatgctgatggcaatggaacgattgactttcctgagttcctcaacctgatggcgcgtaagatgaaggacactgattccgaggaggagcttaaggaggctttccgcgtgtttgacaaggaccagaatggtttcatctcggcagctgaactccgccatgtcatgaccaaccttggtgagaagctgacagacgaggaggtggatgagatgatccgtgaggctgatgtcgatggtgacggccagatcaattacgaagagtttgtgaaggtgatgatggccaagtgagctgtcatactaagctagttctatctcgatgttagcagctagtctagccttgaaaactgacagcagttggtactactgttaagtccagtgaatgaacatattgtgcgctcagggatgttgagaatttcgtttggattttcttgcagttgtgtttcctttgtacaatgtagctttctttccaaattgagatcatga

SEQ ID NO: 2 - DNA, 18 bp, synthetic construct
  source 1..18: mol_type = other DNA
ggcaaggattcaagcaag

SEQ ID NO: 3 - DNA, 21 bp, synthetic construct
  source 1..21: mol_type = other DNA
catcacaataggcaacagagt

SEQ ID NO: 4 - DNA, 18 bp, synthetic construct
  source 1..18: mol_type = other DNA
caacgggaagtccaagcg

SEQ ID NO: 5 - DNA, 18 bp, synthetic construct
  source 1..18: mol_type = other DNA
atctggtcgtcggtgagc

SEQ ID NO: 6 - DNA, 19 bp, synthetic construct
  source 1..19: mol_type = other DNA
gtcgccctccctccttcct

SEQ ID NO: 7 - DNA, 18 bp, synthetic construct
  source 1..18: mol_type = other DNA
accccgcaaaccaatccc

SEQ ID NO: 8 - DNA, 18 bp, synthetic construct
  source 1..18: mol_type = other DNA
ggtggcatatctttgttg

SEQ ID NO: 9 - DNA, 18 bp, synthetic construct
  source 1..18: mol_type = other DNA
tccttgcccctcatcttg

SEQ ID NO: 10 - DNA, 22 bp, synthetic construct
  source 1..22: mol_type = other DNA
tcgatgctcaccctgttgtttg

SEQ ID NO: 11 - DNA, 22 bp, synthetic construct
  source 1..22: mol_type = other DNA
ttggaatgtattggcacaggac

SEQ ID NO: 12 - DNA, 311 bp, unidentified
  source 1..311: mol_type = genomic DNA
actgaggcagagcttcaggatatgatcaatgaagtggatgctgatggcaatggaacgattgactttcctgagttcctcaacctgatggcgcgtaagatgaaggacactgattccgaggaggagcttaaggaggctttccgcgtgtttgacaaggaccagaatggtttcatctcggcagctgaactccgccatgtcatgaccaaccttggtgagaagctgacagacgaggaggtggatgagatgatccgtgaggctgatgtcgatggtgacggccagatcaattacgaagagtttgtgaaggtgatgatggc

SEQ ID NO: 13 - DNA, 22 bp, synthetic construct
  source 1..22: mol_type = other DNA
tttagccctgccttcatacgct

SEQ ID NO: 14 - DNA, 23 bp, synthetic construct
  source 1..23: mol_type = other DNA
catgaacattaaagtgatacgtg

SEQ ID NO: 15 - DNA, 23 bp, unidentified
  source 1..23: mol_type = genomic DNA
gaactgtgatgcgctcgctgggg

SEQ ID NO: 16 - DNA, 23 bp, unidentified
  source 1..23: mol_type = genomic DNA
tcaggatatgatcaatgaagtgg

SEQ ID NO: 17 - DNA, 24 bp, synthetic construct
  source 1..24: mol_type = other DNA
gaattcatcctcacgttcaacacc

SEQ ID NO: 18 - DNA, 21 bp, synthetic construct
  source 1..21: mol_type = other DNA
aaggcgggaaacgacaatctg

SEQ ID NO: 19 - DNA, 21 bp, synthetic construct
  source 1..21: mol_type = other DNA
caaacaaaccattcccatctt

SEQ ID NO: 20 - DNA, 22 bp, synthetic construct
  source 1..22: mol_type = other DNA
ctgctgtcagttttcaaggcta

SEQ ID NO: 21 - DNA, 21 bp, synthetic construct
  source 1..21: mol_type = other DNA
atccaagcaagaaccaaaact

SEQ ID NO: 22 - DNA, 21 bp, synthetic construct
  source 1..21: mol_type = other DNA
cctgagaagaaaggaagagcg

SEQ ID NO: 23 - DNA, 20 bp, synthetic construct
  source 1..20: mol_type = other DNA
agtgggctaaggagcaaggt

SEQ ID NO: 24 - DNA, 21 bp, synthetic construct
  source 1..21: mol_type = other DNA
gccatcatcaccttcacaaac

(19) United States
(12) Patent Application Publication
(10) Pub. No.: US 2026/0055424 A1
(43) Pub. Date: Feb. 26, 2026

(54) GENE TaCaM-3A FOR SEED DORMANCY IN WHEAT AND APPLICATION THEREOF

(71) Applicant: Anhui Agricultural University, Hefei City (CN)

(72) Inventors: Haiping ZHANG, Hefei City (CN); Cheng CHANG, Hefei City (CN); Chuanxi MA, Hefei City (CN); Danian YAO, Hefei City (CN)

(73) Assignee: Anhui Agricultural University, Hefei City, AH (CN)

(21) Appl. No.: 19/372,760

(22) Filed: Oct. 29, 2025

(30) Foreign Application Priority Data
Sep. 16, 2025 (CN) 202511323954.0

(51) Int. Cl. C12N 15/82; C07K 14/415

(52) U.S. Cl. CPC C12N 15/8267; C07K 14/415; C12N 15/8261

(57) ABSTRACT

The present disclosure relates to the field of wheat genetic breeding technology, and provides a gene TaCaM-3A for seed dormancy in wheat and application thereof. The full-length sequence of gene TaCaM-3A is shown in SEQ NO.1. The present disclosure identifies a gene TaCaM-3A that negatively regulating seed dormancy and sprouting resistance. Functional loss of this gene can significantly enhance sprouting resistance of wheat varieties, and increase grain length, grain width, and yield per plant. This gene has the potential to simultaneously improve sprouting resistance and yield of wheat, and has significant breeding application value in wheat sprouting-resistance breeding.

### CROSS-REFERENCE TO RELATED APPLICATION

This application claims priority to Chinese Patent Application No. 202511323954.0, filed on Sep. 16, 2025, which is hereby incorporated by reference in its entirety.

### TECHNICAL FIELD

The present disclosure relates to the field of wheat genetic breeding technology, specifically to a gene TaCaM-3A for seed dormancy in wheat and application thereof.

### BACKGROUND ART

The problem of sprouting caused by continuous overcast and rainy weather before wheat harvest is common in major wheat growing regions around the world, such as China, the United States, Canada, France, the United Kingdom, Australia, South Africa, etc. Sprouting causes wheat quality deterioration, yield decline, and seed vitality decline. If sprouted grains continue to mold and accumulate more toxins, they will lose their edible value.

Seed dormancy is closely related to sprouting resistance. The higher the level of seed dormancy of wheat varieties, the higher the sprouting resistance of the wheat varieties. Therefore, it is particularly important to explore genes related to seed dormancy for breeding new wheat varieties with high resistance to sprouting using techniques such as gene editing and gene pyramiding.

The dormancy of wheat seeds is controlled by multiple genes, including major genes and minor genes. Although multiple dormancy genes, such as TaVp-1, TaMFT, TaSdr, TaMKK3, TaQsd1, Myb10-D, TaGATA1, TaP14K-2A, TaSRO1, and TaPP2C-a6, have been cloned, their practical application value in wheat sprouting-resistance breeding is still unclear, resulting in slow progress in wheat sprouting-resistance breeding and very few wheat varieties that can achieve resistance levels in production, making it difficult to cope with frequent overcast and rainy weather during the harvest period. Therefore, identifying new seed dormancy genes with breeding application value can help accelerate the breeding and promotion of new sprouting-resistance wheat varieties, in order to alleviate the disasters caused by sprouting.

### SUMMARY

The purpose of the present disclosure is to provide a gene TaCaM-3A for seed dormancy in wheat and application thereof. The gene TaCaM-3A has the function of negatively regulating seed dormancy and sprouting-resistance of wheat, providing a gene target having an important breeding value for efficiently improving the sprouting resistance of modern wheat varieties using gene editing technology.

To achieve the above objectives, the present disclosure discloses a gene TaCaM-3A for seed dormancy in wheat, a full-length sequence of the gene is shown in SEQ NO.1.

The present disclosure also provides a method of application of the gene TaCaM-3A for seed dormancy in wheat in regulation of seed dormancy and sprouting resistance.

In some embodiment, the regulation is a negative regulation.

The present disclosure further provides a method for improving sprouting resistance of wheat, including reducing an expression level of gene TaCaM-3A in wheat or deleting function of the gene TaCaM-3A, and where a sequence of the gene TaCaM-3A is shown in SEQ NO. 1.

In some embodiments, reduction of the expression level of the gene TaCaM-3A in wheat or deletion of function of the gene TaCaM-3A is achieved through RNA interference or a gene editing technology.

In some embodiment, the gene editing technology uses a CRISPR/Cas9 system, and an sgRNA target sequence used by the CRISPR/Cas9 system is a sequence as shown in SEQ ID NO.15 or SEQ ID NO.16.

The present disclosure further provides a method for increasing wheat yield, including reducing an expression level of gene TaCaM-3A in wheat or deleting function of the gene TaCaM-3A, and where a sequence of the gene TaCaM-3A is shown in SEQ NO. 1.

In some embodiments, the increasing wheat yield includes increasing grain size, grain weight, or yield per plant.

The advantages and positive effects of the gene TaCaM-3A for seed dormancy in wheat and application thereof described in the present disclosure are as follows:

The present disclosure identifies a gene TaCaM-3A for negatively regulating seed dormancy and sprouting resistance. Functional loss of this gene can significantly enhance sprouting resistance of wheat varieties, and increase grain length, grain width, thousand grain weight, and yield per plant. This gene has the potential to simultaneously improve sprouting resistance and yield of wheat, and has significant breeding application value in wheat sprouting-resistance breeding.

The technical solution of the present disclosure will be further described in detail through the accompanying drawings and embodiments.

### BRIEF DESCRIPTION OF THE DRAWINGS

FIGS. 1A-1B show changes of expression level of gene TaCaM-3A in different seed imbibition period (imbibed for 4 h, 6 h, and 10 h, i.e., the dormancy process is gradually released) in Jing 411 (J411, weak dormancy/susceptible to sprouting) and Hong Mang Chun 21 (HMC21, strong dormancy/sprouting resistance), where FIG. 1A shows transcriptome sequencing and FIG. 1B shows qRT-PCR validation.

FIG. 2A shows the mutation site of the homologous gene of TaCaM (TaCaM-3A/3B/3D) in the gene editing plant cam-ko-2/3/6, and FIG. 2B shows the expression level of the gene TaCaM-3A in the TaCaM-3A overexpression (CaM-OE-2/8/9) and TaCaM RNA interference (cam-rnai-5/8/9) plants, as well as the wild-type Fielder.

FIGS. 3A-3C show the verification of the negative regulation of seed dormancy and sprouting resistance of gene TaCaM-3A using wheat gene editing materials, where FIG. 3A shows the germination percentage of seeds of gene TaCaM edited plants cam-ko-2/3/6 and wild-type KN199 harvested from Hebei and Henan experimental stations in 2025 on the third day of imbibition, FIG. 3B shows the germination phenotype of seeds of gene TaCaM edited plants and wild-type KN199 harvested from Hebei and Henan experimental stations in 2025 on the third day of imbibition, and FIG. 3C shows the germination phenotype of spikes of gene TaCaM edited plants and wild-type KN199 harvested from Hebei and Henan experimental stations in 2025 on the third day of imbibition.

FIGS. 4A-4D show the verification of functions of negative regulation of seed dormancy and sprouting resistance of gene TaCaM-3A by using wheat overexpression and RNA interference materials, where FIG. 4A shows the germination percentage of seeds of gene TaCaM RNA interference plants cam-rnai-5/8/9 and TaCaM-3A overexpression plants CaM-OE-2/8/9 and wild-type Fielder harvested from Hebei and Henan experimental stations in 2025 on the third day of imbibition, FIG. 4B shows the germination phenotype of seeds of gene TaCaMRNA interference plants cam-rnai-5/8/9 and TaCaM-3A overexpression plants CaM-OE-2/8/9 and wild-type Fielder harvested from Hebei experimental station in 2025 on the third day of imbibition, FIG. 4C shows the germination phenotype of seeds of gene TaCaMRNA interference plants cam-rnai-5/8/9 and TaCaM-3A overexpression plants CaM-OE-2/8/9 and wild-type Fielder harvested from Henan experimental station in 2025 on the third day of imbibition, and FIG. 4D shows the germination phenotype of spikes of gene TaCaM RNA interference plants cam-rnai-5/8/9 and TaCaM-3A overexpression plants CaM-OE-2/8/9 and wild-type Fielder harvested from Hebei and Henan experimental stations in 2025 on the third day of imbibition.

FIGS. 5A-5K show comparisons of yield related traits between gene TaCaM edited plants and wild-type KN199, where FIG. 5A shows the comparison of grain size (grain length and grain width) between gene TaCaMedited plants cam-ko-2/3/6 and wild-type KN199 harvested from the field of Hebei in 2025, FIG. 5B shows the comparison of grain size (grain length and grain width) between gene TaCaM edited plants cam-ko-2/3/6 and wild-type KN199 harvested from the field of Henan experimental station in 2025, FIG. 5C shows the comparison of grain length between gene TaCaM edited plants cam-ko-2/3/6 and wild-type KN199 harvested from the field of Hebei and Henan experimental stations in 2025, FIG. 5D shows the grain width, FIG. 5E shows the thousand grain weight, FIG. 5F shows the grain yield per plant, FIG. 5G shows the plant height, FIG. 5H shows the tiller number, FIG. 5I shows the spike length, FIG. 5J shows the plant growth of gene TaCaM edited plants cam-ko-2/3/6 and wild-type KN199 planted in the field of Hebei experimental station in 2025, and FIG. 5K shows the plant growth of gene TaCaM edited plants cam-ko-2/3/6 and wild-type KN199 planted in the field of Henan experimental station in 2025.

FIGS. 6A-6K show comparisons of yield related traits between gene TaCaM edited plants and wild-type KN199, where FIG. 6A shows the comparison of grain size of TaCaM RNA interference plants cam-rnai-5/8/9 and TaCaM-3A overexpression plants CaM-OE-2/8/9 as well as wild-type Fielder harvested from the field of Hebei experimental station in 2025, FIG. 6B shows the comparison of grain size of TaCaM RNA interference plants cam-rnai-5/8/9 and TaCaM-3A overexpression plants CaM-OE-2/8/9 as well as wild-type Fielder harvested from the Henan experimental station in 2025, FIG. 6C shows the comparison of grain length of TaCaM RNA interference plants cam-rnai-5/8/9 and TaCaM-3A overexpression plants CaM-OE-2/8/9 as well as wild-type Fielder harvested from the Hebei and Henan experimental stations in 2025, FIG. 6D shows the grain width, FIG. 6E shows the thousand grain weight, FIG. 6F shows the grain yield per plant, FIG. 6G shows the plant height, FIG. 6H shows the tiller number, FIG. 6I shows the spike length, FIG. 6J shows the plant growth of TaCaM RNA interference plants cam-rnai-5/8/9 and TaCaM-3A overexpression plants CaM-OE-2/8/9 as well as wild-type Fielder planted in the field of Hebei experimental station in 2025, and FIG. 6K shows the plant growth of TaCaMRNA interference plants cam-rnai-5/8/9 and TaCaM-3A overexpression plants CaM-OE-2/8/9 as well as wild-type Fielder planted in the field of Henan experimental station in 2025.

### DETAILED DESCRIPTION OF THE EMBODIMENTS

The following provides further explanation of the technical solution of the present disclosure through the accompanying drawings and embodiments.

Based on the embodiments of the present disclosure, all other embodiments obtained by ordinary skilled in the art without creative labor are within the scope of protection of the present disclosure. The experimental methods without specific conditions specified in the following embodiments are usually determined according to national standards. The experimental instruments, equipment, and reagents not indicated in the following embodiments are all commercially available raw materials.

Unless otherwise defined or specified, all professional and scientific terms used in the present disclosure have the same meanings as those familiar to those skilled in the art. In addition, any methods and materials similar or equivalent to the recorded content can be applied to the method of the present disclosure. It should be noted that the embodiments and features in the embodiments of the present disclosure can be combined with each other without conflict.

### EMBODIMENTS

1. 1) Cloning of gene TaCaM-3A:

The genomic DNA of genome of wheat leaves is extracted, and PCR is performed using a cloning primer set of gene TaCaM-3A. The sequence of the primer set is as follows:

TaCaM-3A-F1 (SEQ ID No. 2):
5′-GGCAAGGATTCAAGCAAG-3′;
TaCaM-3A-R1 (SEQ ID No. 3):
5′-CATCACAATAGGCAACAGAGT-3′;
TaCaM-3A-F2 (SEQ ID No. 4):
5′-CAACGGGAAGTCCAAGCG-3′;
TaCaM-3A-R2 (SEQ ID No. 5):
5′-ATCTGGTCGTCGGTGAGC-3′;
TaCaM-3A-F3 (SEQ ID No. 6):
5′-GTCGCCCTCCCTCCTTCCT-3′;
TaCaM-3A-R3 (SEQ ID No. 7):
5′-ACCCCGCAAACCAATCCC-3′;
TaCaM-3A-F4 (SEQ ID No. 8):
5′-GGTGGCATATCTTTGTTG-3′;
TaCaM-3A-R4 (SEQ ID No. 9):
5′-TCCTTGCCCCTCATCTTG-3′.

The splicing, alignment, and structural analysis of the sequencing results of gene TaCaM-3A are completed using DNAMAN software (https://www.lynnon.com/dnaman.html). The full-length sequence of the gene TaCaM-3A is shown in SEQ ID No.1:
AGCGTCGTCGAGGTCTGGAAGTGTCTGACAAGTGCCCTCTCTGTGCTTTGGA GCCGGAGGACACGTTCCATGCTTTTTTGCAGGTGCCCGCTGGCGGTGGCGCTCTG GCAAACCATGGCGGACCAATGGCGAATCCCGGATGTCGCATCATTCCGCCGGACG GGATCGGAGTGGCTTGCCCAAGCCCTTTGCGATCTGCCGGATATGGAGAGGATGG AGCTCATGATGACTTTGTGGCGATGTTGGTTCGTTCGCAACGAGCTGGTGCACCAC AAGAAGCCGCCGCCGATTGAAGTGTCCAAACGCTTCCTCACCAGCTACGTCGACT CTTTGGTGGGGATTCAGAACAATCCAGAGGCGAGTCTCACCAAGGGGAAGCATGT GGTTGATACGATTGTCCCCAAACGGATCGTGCAACGGGAAGTCCAAGCGCCGCCA GTGCCGCTGCACTGGTCCAGACCAGCCACGGGGTGGACAAAGCTCAACATTGATG GATCCTTTAGTGCCACGGGTGGAGAGGCCGGAGCAGGCATGATCGTTCACAGCGA AACCGGTGATATCATCTTCTCCTCCTGCAGGGAACTACGGATGTGCTCTGAGCCCC TTGAGGCCGAGCTACATGCATGCATGGAGGGCTCAATCTAGCACTGTAGTGGACTC TGTTGCCTATTGTGATGGAAACGGACTGTTCGGTGGCACTACATGCGATATTAGCTC CAGTGCCGGACAGATCTCGTTTTGCTATGCTTGTCGACCAGGTTAGACGCTTGATG CCAGGAGGTAGAGAAATAAAGATGGTTCATGTATGTAGAGAGGAGAATAGGGTAA GCCACTATCTTGCGAACTACGGTAGAGTTCATAAACGCATGATTGTGTGGCTGGGA TCGGGACCGAAGAAGGTCCCAGATTTGTGTAAGGCCGAGGCCCTTTATGTGTGAG AAATGAAATTTCTTTCACCCGCAAAAAAAAGAGATCAAATACATGAAGATAGGCAT AGGCATGTGGACTTTACTTACAACATTATAGTTGCACTTAGGATCTTCTCTTAATTTC CAATATGGTGATGTGGTCTCAACAATTAGATATGGGGAATGCGTCCAAATAATATCA CAACCAATCAACAACCTACATCGTGAGTCTATTGTCCAAACAAGGCCCTTACCGTC ACATGGGGTTACGTCGATTATTAAGGATTTCTCCAAGCCAACACAATTGGCGTTCA GTGACTACACCTCATGATTCCTTGTTAGGTCTTTTCGGCTACCGAACCAATCCTTAC TTTCATAGGCGTTGGTGTAACCTTCTACGTGTCCCTGCACTTTATTTCCTCCTACGTT TTTTGGATGCAATGGAAAAAAGTGCTCCCTGTGGTTTTTTTTGGGTGGATCGGGCA TGACGAAGGTCCGGATACCCGTAAACCTCTCCTACATTTGGTTTTGGTTTGTGTGAT TTTAGACTCCAGTATGGTTTGGACAAATTTAATGACCCACGTTGACAAAATATTTGG ACCATTCGGTCTACACATTTAAAGGCGATTTGGTGAACCACATTGAAGTTGCCGTT ACAAGGGTGAATCGTTTTGTTGGATGCGTTATACCGTTCGCCAGCTAGGGTTGACA TTTGTGAACCGCCTAGATTGCAGTTTAAGTGTGCTTGGGGACGGCAGTTTTACAAA AGGCATGGCATTTTATTTATTTTAGTGCATTTTCATCTAAAAACTGCCACCATCAAAT CTTGATCGTGTGGCGTGTGGAGGGGTTGCTGGAGGGATGTTCCTGGCGACTGTTC GCCAGGTAATAACATTGCCAAAAAGAAAAAAAAAAGCAGTAGAGCGTGATTTTGA GCCGCGGTACAAAGGTACCCGTAGCAGGTTGCCCCTATCCGTATCCGGTCGCCTAA ATATTTTCCTCCCTGTCGCCCTCCCTCCTTCCTTTCCTCCACTCGCGCTTCGCTCTCA GATCCCTTCTCCTCCGTCTCGCCCGCGTGTTGGGGTAGGAGCCATGGCGGACCAGC TCACCGACGACCAGATCGCCGAGTTCAAGGAGGCCTTCAGCCTCTTCGACAAGGA CGGAGACGGTTCGATCCCTTCTCTGTTCTCTTTCAACATGTACTACTATATGATTTGC ACGACTCCATTCGATGTGCGCGCACTCGCTGTGGTTGATGCGGCGCCTTGCACTGG CGCGTGGATGTGTTGGGTGGGAGATCTGATCGACGTAGCGTATATGCGTTTGCTGG TCCGAGTGGATCGCGGCGGGATTGGTTTGCGGGGTGCTCAAGATGCAGTAGATCTT GCATTCCTTTTCTCCACTCCTTGTGGATTCTGGTTCCATACATTCTTGTCTTGCCAGC GCTATCAACCCGTCACCGTCCAGTGTTCTGGTATTGCTGCGCCTTATGCTCGTCCGT TCCGGCTGATCGCTGAGCTGTCTGTCATAGCGAAGGCTTCAGCCGGCTGTTTGGTC GTTCCTTCAGATCTCTTTCAGTGGAGCAACTGATGACCTTGCAAAGTTCCAAAAAA AAAAGGTTCTACTAGGAAGAGATTGAATTATGAGAGAAGGTGGCATCATGGGGTT GCTGCGGTTAGCATGGGTCTACCGCCCACACATCTAGTGGTGTCTGCAGCTTAAGA AAACCGAGGGTGCAGCATGGGTGATCACACAGCATGGCCGGTGGCACTGTTGCTG CTGAGTAGGTTGCTAGAGCATGGTGGGTCACTAGTCATCAGTCTCTACTCTAGATTT AGCTGGAACAGCAGTGACGAGGTGTGCTGCGAAGTGGTGTTACCGTGTTAGGGTT ATAGGATCATGTAATACCCATAATTCTGTACTTGTATGTTAGTAATGGGGAACTACTA CTGACTTGGAATTTATCTAGGCTTTTGGGTCTTTTTCTATGGATGACATGGGCATAG AAGTGGGATAGCATGATTAAATTTTGCGTTGCTCAAACTGCACATTTTCTTCTCAGT GAATGCAACATCGGAGTTTCTCTGGTTTTGCCTATCATGCTATGGTGGTGCTAGTGT GAGACTGATAACATGGAGAATGCATGTGATGGCTTGAAAAGTTATTGGTATTGATG TTTACTGATTGTATGAAACATTAGTTGAAGTAATTTACATGTTACCAGCTTACTGATT GACAGAAAATATATCTGTAAGTTGCTTACTGATTGACATGCAACCTATTACAGCCAA GCAAATCTTTGCTATAATAGGTTGCATGTCTTACTCTTCTATTTATTTGGGCTTTGGT ACTGATCAAGTCGATGGAATGTTTACCCAAATACTTTTCTGGATAGTTGTGTTCCAG ATAGTCAAGCCTTGAATGTTCTTATGAAATTTCATCATTGTCCACTTAAACTGTATTT GTATTTATTTGCTACCCTTGAATGATTTCATGCACCCAAAATTCCAGATGAAGAAAG GGACTGTAAGATTTGTTCTGATGGACCCAATCATTGGTCCATATTGTCTTTTGCATTA GCATAGCTGAAATGATCTCTAATGGTTAAATAAATGTAAATAATATTTGAGTCCAAC TGTCCATTGAAAACCCCCAAGTTCGTTATGTTCTTGTACTGCACATCCATGTGCAAA TGTGGCTTATAAATTTCTATAAGATCATTTTGAAGATTCGCCCATTACTGGCACATTT CATATAGAATACGTGCAGCATTTTACATATGATATGATGATTTTATTGCACAATTACTT GTGTTTTTCTGCATACTTGTGGGTTTCTGTATTCCCATCTAATAGTCAGTGTTTCAAG TGGGCAAGTCGAATCAAGTTGCACCTGACTGGCCAGGTGACTAGTCTGACTATAG ATGCTGACTAGTCAGTGACTATTTGACAATTAGCTAGGCTTGCATAGTGAGGCCATT TAGACAGCTGGCAATAGGTTTTCAAGTCATACTTGGTGTGTCTGGTTTGAGCCCAA GCCAAACCCCACCAAAAATTTGGCTTAGGTTTTGTCTGCCCATGTTTTGGCCACTG CTGGCAAGAAAATTGTAAGTGGGCTAAGGGGCATGGACGCATGGTCATGCCAAAT TTTTGTCAGCCATCCAAGCAAGAACCAAAATTCAGTCATAACCAAAACAATCATTA GGTTTGATTTGGTTGCAAACAAACCATTCCCATCTTCCATGTTAAGACCTCTTTTAT TGTGCACAGAATTGAATTGCTGACATAAATCTCCGGGGCATTTCATTCTAGCACAAT ATTACCATTGTATATATATGAGCAATGCTACACCTACGTAAAGCTGCCATACGTGATT AACGTAACAGCCTAGGTGGCATATCTTTGTTGGANNNNNNNNNNNNNNNNNNNN NNNNNNNNNNNNNNNNNNNNNNNNNNNNNNNNNNNNNNNNNNNNNNNNNNNN NNNNNNNNNNNNNNNNNNNNNNNNNNNNNNNNNNNNNNNNNNNNNNNNNNNN NNNNNNNNNNNNNNNNNNNNNNNNNNNNNNNNNNNNNNNNNNNNNNNNNNNN NNNNNNNNNNNNNNNNNNNNNNNNNNNNNNNNNNNNNNNNNNNNNGTTTACGT AGGTTTAGCATTTTCGTGTATATATTTGTAGCGTTTTTGTATATATATTTGTAGAAGAA GTTTAAATTGTTAGACTAAAAAACTGTCTGCCTCCTGAACAGGCTGCATCACCACC AAGGAGCTTGGAACTGTGATGCGCTCGCTGGGGCAGAACCCCACTGAGGCAGAG CTTCAGGATATGATCAATGAAGTGGATGCTGATGGCAATGGAACGATTGACTTTCCT GAGTTCCTCAACCTGATGGCGCGTAAGATGAAGGACACTGATTCCGAGGAGGAGC TTAAGGAGGCTTTCCGCGTGTTTGACAAGGACCAGAATGGTTTCATCTCGGCAGCT GAACTCCGCCATGTCATGACCAACCTTGGTGAGAAGCTGACAGACGAGGAGGTGG ATGAGATGATCCGTGAGGCTGATGTCGATGGTGACGGCCAGATCAATTACGAAGAG TTTGTGAAGGTGATGATGGCCAAGTGAGCTGTCATACTAAGCTAGTTCTATCTCGAT GTTAGCAGCTAGTCTAGCCTTGAAAACTGACAGCAGTTGGTACTACTGTTAAGTCC AGTGAATGAACATATTGTGCGCTCAGGGATGTTGAGAATTTCGTTTGGATTTTCTTG CAGTTGTGTTTCCTTTGTACAATGTAGCTTTCTTTCCAAATTGAGATCATGA. N refers to an unknown or uncertain nucleobase in the reference genome sequence of the Chinese Spring (CS) background.

### 2) TaCaM-3A is Overexpressed in Wheat Variety Fielder to Obtain Transgenic Wheat Plants:

The exon sequence of TaCaM-3A is connected to the UbiF1899 vector (which is deputed by Sangon Biotech (Shanghai) Co., Ltd. to construct) to obtain the correct recombinant vector Ubi: TaCaM-3A. Then these vectors are transformed into the wheat variety Fielder. When T₀-generation transgenic seedlings grow to 30 cm, leaves of the seedlings are removed and DNA of the leaves are extracted according to the wheat leaf genome DNA extraction method. The specific primers Ubi1899-F (SEQ ID No. 10): TCGATGCTCACCCTGTTGTTTG and TaCaM-3A-OE-R (SEQ ID No. 11): TTGGAATGTATTGACAGGAC are used to detect whether the overexpression vector has been successfully transferred into the receptor material Fielder. Then the seeds of T₁-generation positive plants are harvested. The qRT-PCR method is used to detect the relative expression level of gene TaCaM-3A in the seeds of overexpressing plants, and positive plants with relatively high expression level are selected for subsequent experiments. This study adopts homozygous T₂-generation transgenic plants for determination tests of seed dormancy and sprouting resistance.

### 3) the TaCaM is Knocked Down in Wheat Variety Fielder to Obtain Wheat TaCaM Interference Plants:

The gene fragment (SEQ ID No.12: ACTGAGGCAGAGCTTCAGGATATGATCAATGAAGTGGATGCTGATGGCAATGGAACG ATTGACTTTCCTGAGTTCCTCAACCTGATGGCGCGTAAGATGAAGGACACTGATTCCG AGGAGGAGCTTAAGGAGGCTTTCCGCGTGTTTGACAAGGACCAGAATGGTTTCATCT CGGCAGCTGAACTCCGCCATGTCATGACCAACCTTGGTGAGAAGCTGACAGACGAG GAGGTGGATGAGATGATCCGTGAGGCTGATGTCGATGGTGACGGCCAGATCAATTAC GAAGAGTTTGTGAAGGTGATGATGGC) is inserted into two polyclonal sites upstream and downstream of the pWMB006 vector, respectively. After completion of the connection and sequencing validation, large fragments are recovered using double digests Hind III and EcoR I, and then the large fragments are connected to pCAMBIA3301 vector having the same digestion characteristics. The TaCaM-RNAi vector is confirmed to be constructed correctly by using sequencing verification. After the TaCaM-RNAi vector is correctly constructed, the recombinant plasmid is transformed into the wheat variety Fielder. When the T₀-generation seedlings grow to 30 cm, DNA is extracted from the leaves using the wheat leaf genome DNA extraction method, and Ubi1899F (SEQ ID No.13): TTTAGCCCTGCCTTCATACGCT and OsintronR (SEQ ID No.14): CATGAACATTAAAGTGATACGTG are used to detect whether the TaCaM-RNAi vector has been successfully transferred into the receptor material Fielder. After confirming the successful transfer of the vector, seeds of T₁-generation positive plants are collected, the wheat grain RNA extraction method is used to extract the RNA, and qRT-PCR is used to select plants with low expression levels for subsequent experiments.

### 4) the TaCaM is Edited in Wheat Variety KN199 to Obtain Wheat Knockout Plants:

Two sgRNAs on the second exon of TaCaM are designed using the CRISPR direct tool. After the target sequences of TaCaM (gRNA1 (SEQ ID No. 15): GAACTGTGATGCTCGCTGGGG and gRNA2 (SEQ ID No. 16): TCAGGATATGAAGTGG) are introduced into the Cas9 vector, the wheat variety KN199 is transformed and TO-generation transgenic positive seedlings is obtained by sequencing. After the genome DNA of gene TaCaM edited plants is extracted according to the wheat leaf genome DNA extraction method, TaU3 and Cehind primers are first used to detect the successful transfer of CRISPR/Cas9 vector into the receptor material KN199. Then, specific primers TaCaM-3A-Cas9-F/R, TaCaM-3B-Cas9-F/R, and TaCaM-3D-Cas9-F/R are designed for the three homologous genes TaCaM-3A/3B/3D according to the target sites to detect changes in the target sequence of transgenic plants;

TaU3F (SEQ ID No. 17):
GAATTCATCCTCACGTTCAACACC;
CeHindR (SEQ ID No. 18):
AAGGCGGGAAACGACAATCTG;
TaCaM-3A-Cas9-F (SEQ ID No. 19):
CAAACAAACCATTCCCATCTT;
TaCaM-3A-Cas9-R (SEQ ID No. 20):
CTGCTGTCAGTTTTCAAGGCTA;
TaCaM-3B-Cas9-F (SEQ ID No. 21):
ATCCAAGCAAGAACCAAAACT;
TaCaM-3B-Cas9-R (SEQ ID No. 22):
CCTGAGAAGAAAGGAAGAGCG;
TaCaM-3D-Cas9-F (SEQ ID No. 23):
AGTGGGCTAAGGAGCAAGGT;
TaCaM-3D-Cas9-R (SEQ ID No. 24):
GCCATCATCACCTTCACAAAC.

### 5) Planting of Experimental Materials:

J411, HMC21, Fielder, KN199, overexpression, RNA interference, and edited plants have been planted in experimental foundations in Henan and Hebei in the autumn of 2025.

### 6) Phenotypic Determination of Seed Dormancy and Sprouting Resistance:

The present disclosure uses seed Germination Percentage (GP) to measure the levels of seed dormancy and sprouting resistance of wheat. Thirty intact seeds of J411, HMC21, TaCaM-3A overexpression, TaCaMRNA interference, and edited wheat plants are selected, respectively. They are evenly placed in a 90 mm diameter culture dish with the ventral sulcus facing downwards, 10 mL sterile water is added into the culture dish and is incubated for 3 days at 20° C.±2° C. for 14 h (daytime)/10 h (nighttime). The number of germinated seeds on the third day of the germination test is recorded. All the seed germination tests are conducted three times. The GP of seeds is calculated according to the following formula: GP=number of germinated seeds/total number of seeds*100%. Phenotypic data organization is completed by Excel software.

7) Determination of Yield Related Traits of TaCaM-3A Overexpression, Gene TaCaM editing, and Interference Plants:

When wheat grains mature, five representative plants of each genotype are selected for determination of related agronomic traits. The grain length and width, number of grains, and thousand grain weight of wheat are measured using a grain weight meter and a Wanshen SC-G automatic grain analyzer (Hangzhou Wanshen detecting Technology Co., Ltd.). The detection is repeated for 3 times, and the average of the detections are selected as the final result. The number of grains of spike (grains per spike) refers to the number of grains in each individual spike, and the average of number of grains in five plants is taken as the final result. The yield per plant=(grains per spike×number of effective tillers×thousand grain weight)+1000. The plant height is measured using a straightedge measurement method (unit: centimeter) from the base to the top of the highest spikelet, excluding awns. Five plants are counted and the average value of the plant height is taken. Five plants are counted and the average value of the tillers of the plants is taken as the number of effective tillers. The single spike length (spike length) is measured from the base of the first spikelet to the tip of the last spikelet, excluding awns.

### 2. Experimental Results:

### 1) Expression Level of Gene TaCaM-3A:

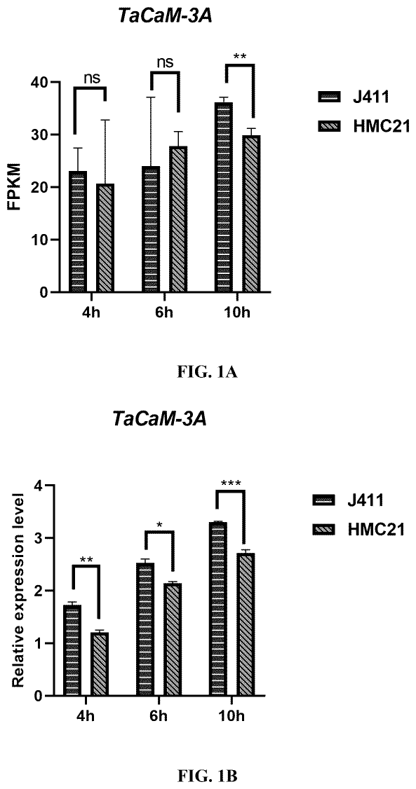

Through transcriptome sequencing, one calcium ion (Ca²⁺) signal receptor gene TaCaM-3A (encoding calmodulin protein) is identified. As shown in FIG. 1a, it is differentially expressed in the seeds of the wheat variety “Hong Mang Chun 21” (abbreviated as HMC21) with strong dormancy/sprouting resistance and the wheat variety Jing 411 (abbreviated as J411) with weak dormancy/sensitive to sprouting, with significantly higher expression levels in J411 seeds than in HMC21 seeds. As shown in FIG. 1b, qRT-PCR technology is used to verify that seeds of J411 and HMC21 are not germinated at 4 h and 6 h of seed imbibition. When the seeds are imbibed for 10 h, seeds of J411 are sprouted, while seeds of HMC21 are remained in a dormant state and are not sprouted. The expression level of gene TaCaM-3A is negatively related with the dormancy depth of wheat seeds.

### 2) Functional Verification of Gene TaCaM-3A:

By constructing plants of overexpression, RNA interference (knock down), and gene editing (knock out), the gene function is verified combining with determination of germination percentage:

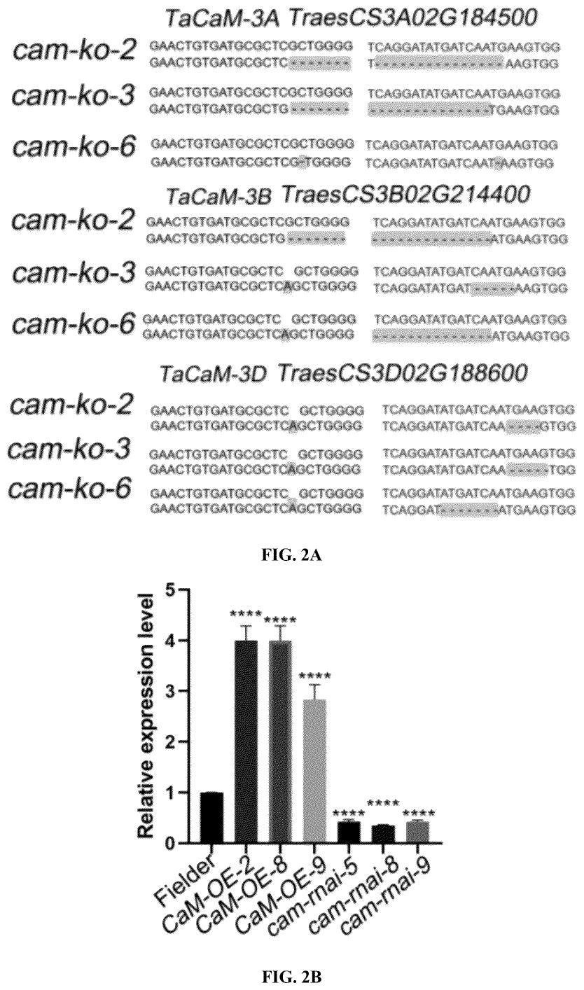

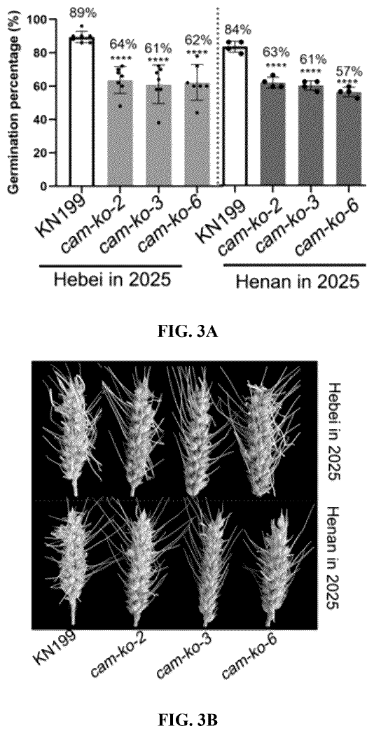

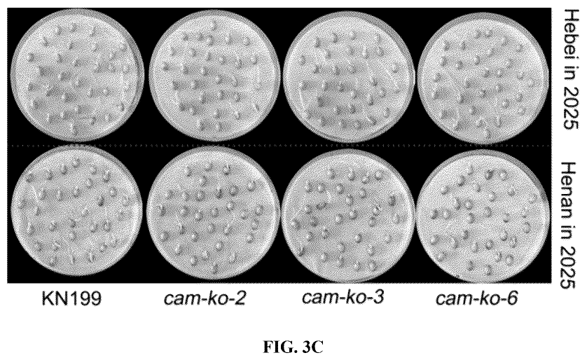

(1) Gene edited plant (cam-ko-2/3/6): Mutation in TaCaM-3A/3B/3D is confirmed by target sequencing (FIG. 2a). The determination of germination percentage of seeds in Hebei and Henan experimental stations in 2025 shows that the germination percentage of the edited plant is significantly lower than that of the wild-type KN199, and the germination phenotype of seeds and spikes is also significantly delayed than that of the wild-type KN199 (FIGS. 3a-3c).

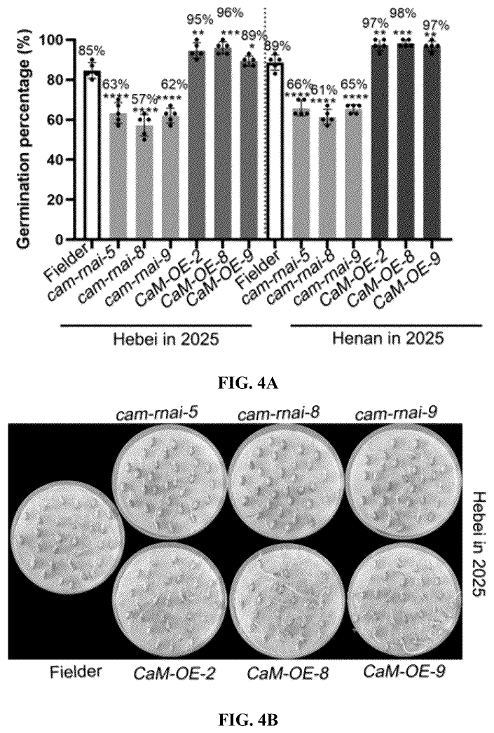

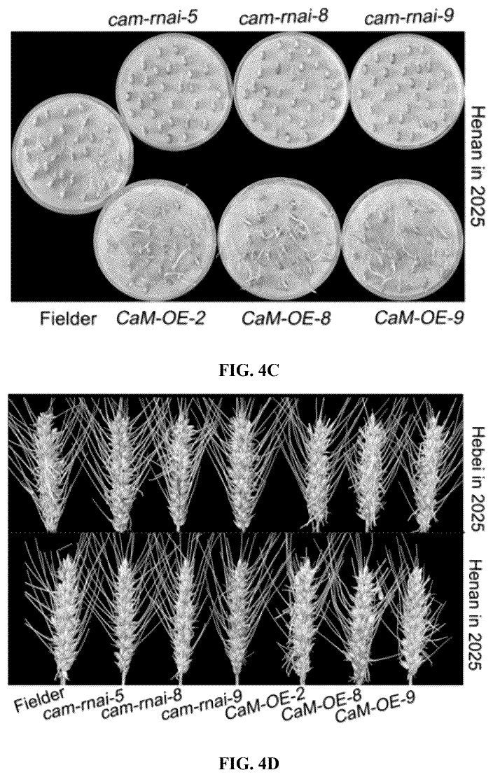

(2) RNA interference plant (cam-rnai-5/8/9): significant decrease in the expression level of TaCaM-3A is confirmed by qRT-PCR (FIG. 2b): The germination percentages of the experimental foundations in Hebei and Henan show that the germination percentage of the interference plant is significantly lower than that of the wild-type Fielder, and the germination phenotype of the interference plant is delayed than that of the wild-type Fielder (FIGS. 4a to 4d).

(3) Overexpression plant (CaM-OE-2/8/9): significant increase in the expression level of TaCaM-3A is confirmed by qRT-PCR (FIG. 2b). The determination of germination percentage shows that the germination percentage of the overexpression plant is significantly higher than that of the wild-type Fielder, and the overexpression plant sprouts earlier than the wild-type Fielder (FIGS. 4a to 4d).

The germination of seeds and spikes in gene TaCaM edited plants and RNA interference plants is significantly delayed than that of TaCaM-3A overexpressing plants and wild-type KN199 and Fielder. It has been confirmed that the gene TaCaM-3A has the function of negatively regulating seed dormancy and sprouting resistance of wheat.

3) The impact of functional deficiency of gene TaCaM-3A on yield related traits in wheat:

The determination of yield related traits (grain length, grain width, thousand grain weight, yield per plant, etc.) in gene edited plants, RNA interference plants, and overexpression plants shows that:

### (1) Gene Edited Plants:

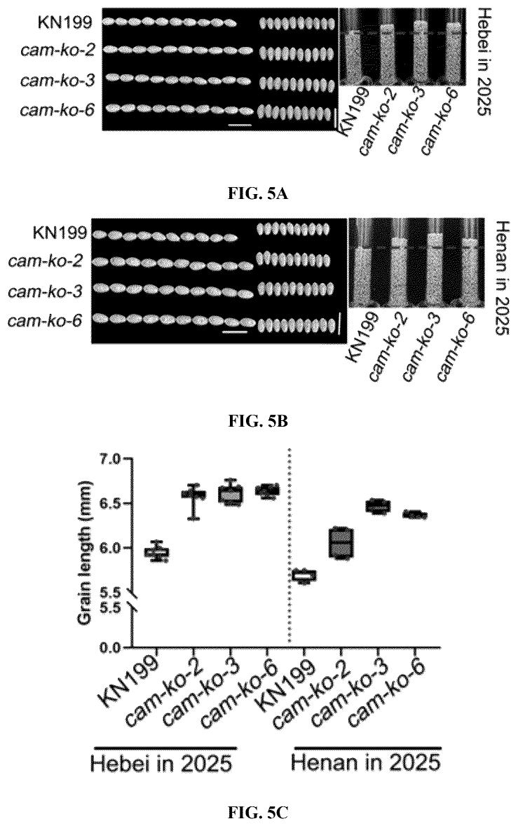

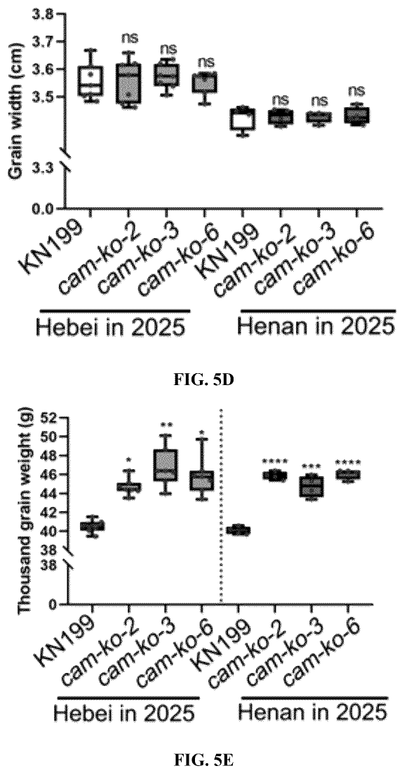

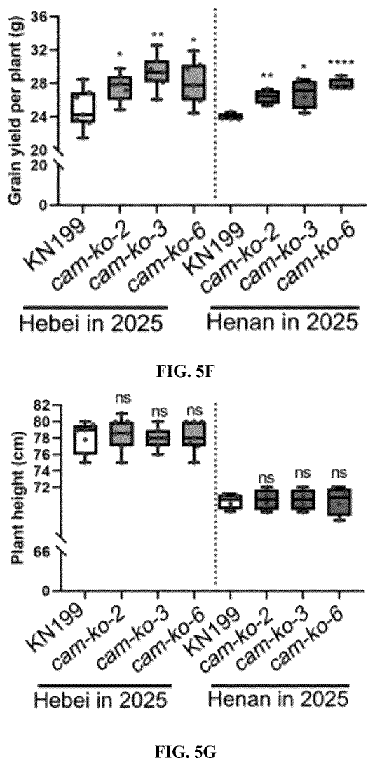

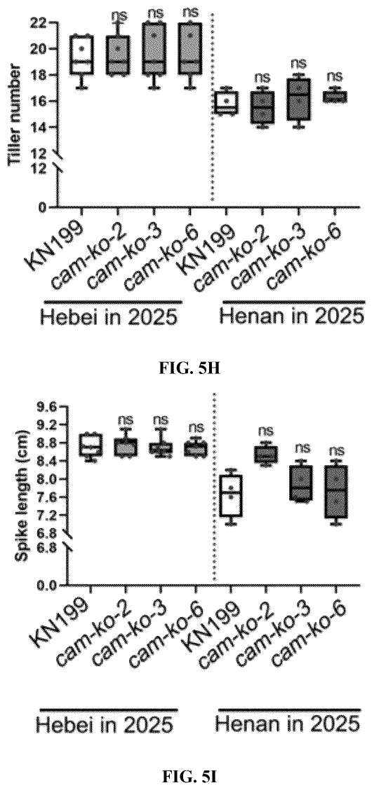

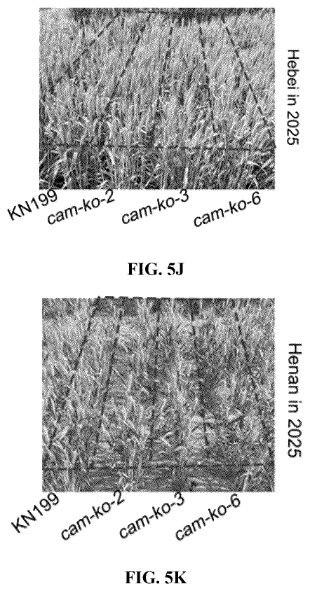

The visual comparison of grain size between Hebei and Henan experimental stations in 2025 shows that the edited plants have larger grains (FIGS. 5a and 5b).

Quantitative analysis shows that the grain length, grain width, thousand grain weight, and yield per plant of the edited plant are significantly higher than those of the wild type (FIGS. 5c to 5f). However, there are no significant differences in traits such as plant height, number of effective tillers, and spike length between the gene edited plant and the wild-type plant (FIGS. 5g to 5i).

The plant growth in the field shows that no significant adverse differences are in plant architecture between the edited plant and the wild-type plant (FIGS. 5j and 5k).

### (2) RNA Interference Plants:

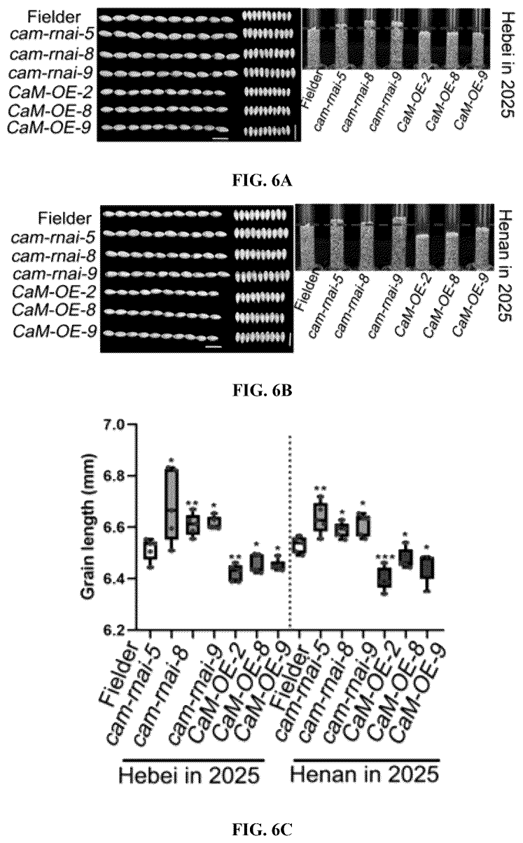

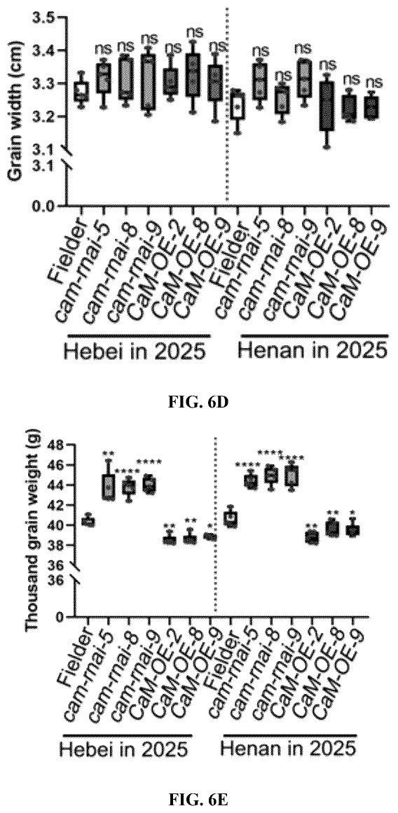

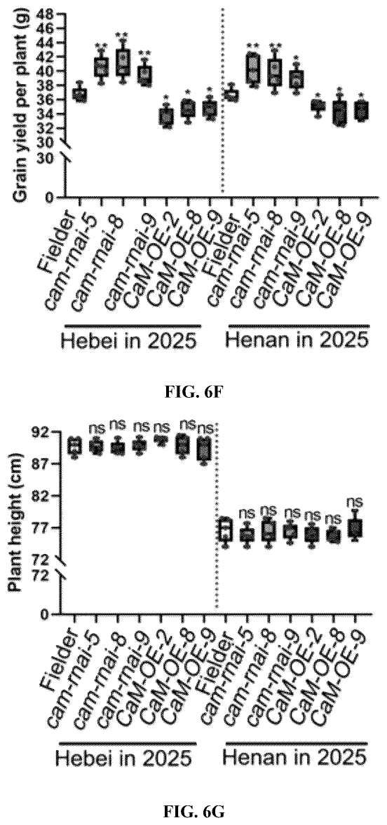

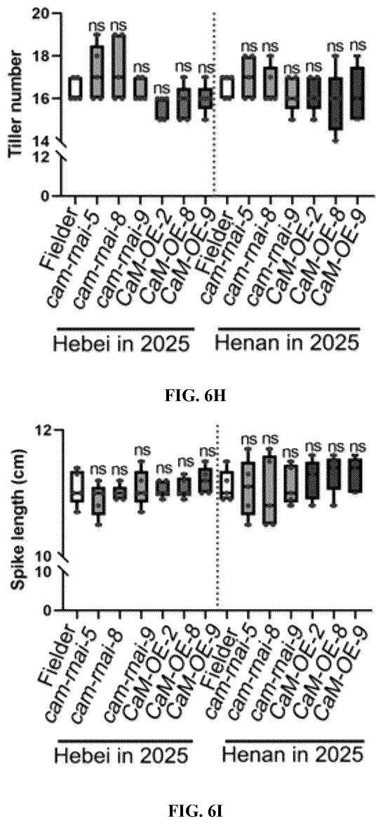

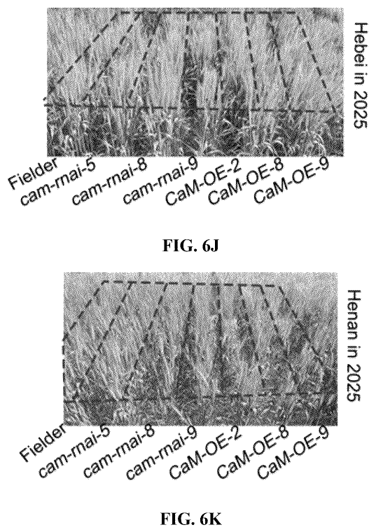

The visual comparison of grain size shows that the grain of the interference plant is larger than that of the wild-type plant (FIGS. 6a and 6b).

Quantitative analysis shows that the grain length, grain width, thousand grain weight, and yield per plant of the interference plant are significantly higher than those of the wild type (FIGS. 6c to 6f). However, there are no significant differences in traits such as plant height, number of tillers, and spike length among the groups (FIGS. 6g to 6i).

The plant growth in the field shows that no significant adverse differences are in plant architecture between the interference plant and the wild-type plant (FIGS. 6j and 6k).

### (3) Overexpression Plants:

The visual comparison of grain size shows that the grain of the interference plant is smaller than that of the wild type (FIGS. 6a and 6b).

Quantitative analysis shows that the grain length, grain width, thousand grain weight, and yield per plant of the overexpression plant are significantly lower than those of the wild type (FIGS. 6c to 6f). However, there are no significant differences in traits such as plant height, number of tillers, and spike length among the groups (FIGS. 6g to 6i).

The plant growth in the field shows that no significant adverse differences are in plant architecture between the overexpression plant and the wild type (FIGS. 6j and 6k).

The functional loss (knocking down/knocking out) of gene TaCaM-3A can significantly improve yield related traits of wheat, increase grain size, grain weight, and yield per plant, and has no adverse effects on plant height, spike length, and architecture.

In summary, the present disclosure has identified a gene TaCaM-3A that negatively regulates seed dormancy and sprouting resistance of wheat. The functional loss of this gene not only significantly enhances sprouting resistance in wheat varieties, but also increases grain length, grain width, and yield per plant. It has the potential to simultaneously improve sprouting resistance and yield in wheat, and has significant breeding application value in sprouting resistance breeding of wheat. These research results also provide gene targets having important breeding values for efficiently improving sprouting resistance in modern wheat varieties using gene editing technology.

Finally, it should be noted that the above embodiments are only used to illustrate the technical solution of the present disclosure and not to limit it. Although the present disclosure has been described in detail with reference to the preferred embodiments, ordinary skilled in the art should understand that they can still modify or equivalently replace the technical solution of the present disclosure, and these modifications or equivalent replacements cannot make the modified technical solution deviate from the spirit and scope of the technical solution of the present disclosure.

This application contains a Sequence Listing XML as a separate part of the disclosure, which presents nucleotide and/or amino acid sequences and associated information using the symbols and format in accordance with the requirements of 37 CFR-1.831-1.835. The XML file named CNUS-SZ-U-66-2025_SEQUENCES.xml, created Oct. 29, 2025, 26,948 bytes in size, is submitted herewith and is incorporated by reference in its entirety.

What is claimed is:

1. A TaCaM-3A gene for seed dormancy in wheat, wherein the TaCaM-3A gene full-length sequence comprises SEQ NO. 1.

2. A method of application of the TaCaM-3A gene for seed dormancy in wheat according to claim 1 comprising regulating seed dormancy and sprouting resistance with the TaCaM-3A gene.

3. The method of application according to claim 2, wherein the regulation is a negative regulation.

4. A method for improving sprouting resistance of wheat, comprising reducing an expression level of TaCaM-3A gene in wheat or deleting function of the TaCaM-3A gene, and wherein the TaCaM-3A gene comprises the sequence of SEQ NO. 1.

5. The method according to claim 4, wherein reduction of the expression level of the TaCaM-3A gene in wheat or deletion of function of the TaCaM-3A gene is achieved through RNA interference or a gene editing technology.

6. The method according to claim 5, wherein the gene editing technology uses a CRISPR/Cas9 system, and an sgRNA target sequence used by the CRISPR/Cas9 system comprises SEQ ID NO.15 or SEQ ID NO.16.

7. A method for increasing wheat yield, comprising reducing an expression level of TaCaM-3A gene in wheat or deleting function of the TaCaM-3A gene, and wherein the TaCaM-3A gene comprises the sequence of SEQ NO. 1.

8. The method according to claim 7, wherein the increasing wheat yield comprises increasing grain size, grain weight, or yield per plant.
